(19) United States
(12) Patent Application Publication
(10) Pub. No.: US 2020/0077714 A1
(43) Pub. Date: Mar. 12, 2020

(54) APPARATUS AND METHODS FOR VAPORIZING AN HERB

(71) Applicant: ZHENGNAN DONG, Richmond Hill (CA)

(72) Inventor: ZHENGNAN DONG, Richmond Hill (CA)

(21) Appl. No.: 16/685,539

(22) Filed: Nov. 15, 2019

Related U.S. Application Data
(63) Continuation-in-part of application No. 16/034,709, filed on Jul. 13, 2018.

(30) Foreign Application Priority Data
Jul. 12, 2019 (CA) 3049533

(51) Int. Cl. A24F 47/00; A61M 11/04

(52) U.S. Cl. CPC A24F 47/008; A61M 16/1075; A61M 11/042

(57) ABSTRACT

Disclosed is an assembly for use in an herb vaporizer. The assembly includes a casing having at least one casing air passageway. The assembly also includes a first grinding element partially defining a grinding chamber and a second grinding element, opposed to said first grinding element, further defining the grinding chamber and rotatably mounted such that contents of the grinding chamber are agitated by rotation of the second grinding element. The assembly also includes a heater within the casing operatively associated with the grinding chamber, and being electrically coupled to a threaded connector providing an electric connection from a battery unit. The threaded connector projects from the casing for mating with an external battery unit.

### CROSS-REFERENCE TO RELATED APPLICATIONS

This application is a continuation-in-part application of U.S. patent application Ser. No. 16/034,709, filed Jul. 13, 2018, the entire contents of which are incorporated herein by reference.

This application also claims the benefit of, and priority from, Canadian Patent Application No. 3049533, filed Jul. 12, 2019, the entire contents of which are incorporated herein by reference.

### FIELD

This disclosure relates generally to apparatus and methods for vaporizing an herb.

### BACKGROUND

Electronic vaporization devices (also known as electronic-cigarettes or vaping devices) utilize electric power to heat liquids or herbs to create a vapor which users may inhale. In the industry, vaporizing is sometimes referred to as atomizing. A limited number of vaporization devices are able to be used with herbs and, therefore, new vaporization apparatus and methods for use with herbs are desirable.

### SUMMARY

In accordance with an aspect of the present disclosure, there is provided an assembly for use in an herb vaporizer comprising a casing having at least one casing air passageway; a first grinding element partially defining a grinding chamber; a second grinding element, opposed to the first grinding element, further defining the grinding chamber and rotatably mounted such that contents of the grinding chamber are agitated by rotation of the second grinding element; a threaded connector projecting from the casing for mating with an external battery unit so as to provide an electrical connection from the battery unit to the threaded connector; and a heater within the casing operatively associated with the grinding chamber, the heater being electrically coupled to the threaded connector.

In one embodiment, the second grinding element has an annular wall with at least one air passageway, and the annular wall is aligned with the at least one casing air passageway such that rotation of the second grinding element rotates the at least one air passageway of the second grinding element into and out of alignment with the at least one casing air passageway, whereby selective rotation of the second grinding element controls airflow through the at least one casing air passageway and rotation of the second grinding element agitates the contents of the grinding chamber.

In one embodiment, the first grinding element has a first annular wall and the second grinding element has a second annular wall, and wherein the second annular wall at least partially overlaps with the first annular wall to define the grinding chamber between the first and second grinding elements.

In one embodiment, the heater abuts a face of the first grinding element external to the grinding chamber.

In one embodiment, the assembly also comprises an insulator within the casing and having an insulator wall defining at least one insulator air passageway, and wherein the at least one insulator air passageway and the at least one casing air passageway are in fluid communication.

In one embodiment, the insulator has an insulator floor, and the heater comprises a heating element sandwiched between the insulator floor and the face of the first grinding element.

In one embodiment, the assembly comprises a grinding element magnet fixed to the second grinding element for releasably securing the second grinding element to the insulator.

In one embodiment, the assembly comprises an insulator magnet fixed to the insulator for coupling with the grinding element magnet to releasably secure the second grinding element to the insulator.

In one embodiment, one of the first annular wall of the first grinding element and the insulator wall has a keyway and another of the first annular wall of the first grinding element and the insulator wall has a corresponding key, and wherein the key is received in the keyway to mount the first grinding element to the insulator.

In one embodiment, the heating element has positive and negative terminals, and wherein the positive and negative terminals extend through the insulator floor and couple to the threaded connector.

In one embodiment, the second grinding element has a disc-shaped wall defining an end of the grinding chamber, the disc-shaped wall having an axially directed opening, and wherein the assembly further comprises a mouthpiece in fluid communication with the axially directed opening.

In one embodiment, the assembly comprises a filter interposed between the disc-shaped wall and the mouthpiece for filtering air flowing from the grinding chamber to the mouthpiece.

In another aspect of the disclosure, there is provided an herb vaporizer comprising a control unit coupled to a battery unit; a casing having at least one casing air passageway; a first grinding element partially defining a grinding chamber; a second grinding element, opposed to the first grinding element, further defining the grinding chamber and rotatably mounted such that contents of the grinding chamber are agitated by rotation of the second grinding element; a heater within the casing operatively associated with the grinding chamber, the heater being electrically coupled to the control unit to provide an electrical connection from the battery unit to the heater; and wherein the second grinding element has an annular wall with at least one air passageway, the annular wall aligned with the at least one casing air passageway such that rotation of the second grinding element rotates the at least one air passageway of the second grinding element into and out of alignment with the at least one casing air passageway, whereby selective rotation of the second grinding element controls airflow through the at least one casing air passageway and rotation of the second grinding element agitates the contents of the grinding chamber.

In yet another aspect of the disclosure, there is provided a method for using an herb vaporizer having a casing with at least one casing air passageway, the method comprising: providing an herb to a grinding chamber of the herb vaporizer, the grinding chamber defined by a first grinding element and a second grinding element, the second grinding element opposed to the first grinding element and rotatably mounted such that the herb is agitated by rotation of the second grinding element, and wherein the second grinding element has an air passageway which, by rotating the second grinding element, is selectively positioned in fluid communication with the at least one casing air passageway; applying heat to the grinding chamber using a heater operatively associated with the grinding chamber; and rotating the second grinding element to control an air flow to the grinding chamber and to further agitate the herb.

In one embodiment, the second grinding element has an annular wall with the air passageway such that rotating the second grinding element rotates the air passageway of the circumferential wall into and out of alignment with the at least one casing air passageway, thereby selectively controlling airflow through the at least one casing air passageway.

Other aspects, features, and embodiments of the present disclosure will become apparent to those of ordinary skill in the art upon review of the following description of specific embodiments in conjunction with the accompanying figures.

### BRIEF DESCRIPTION OF DRAWINGS

In the figures, which illustrate, by way of example only, embodiments of the present disclosure:

FIG. 1 is a bottom-up perspective view of an example embodiment of an herb holding assembly for use in a vaporizer;

FIG. 2 is a top-down exploded view of an upper member of the assembly of FIG. 1;

FIG. 3 is a bottom-up exploded view of the upper member of FIG. 2;

FIG. 4A is a top-down perspective view of the upper member of FIG. 2;

FIG. 4B is a bottom-up perspective view of the upper member of FIG. 2;

FIG. 4C is a side view of the upper member of FIG. 2;

FIG. 4D is a top view of the upper member of FIG. 2;

FIG. 4E is a bottom view of the upper member of FIG. 2;

FIG. 4F is a cross-section view of the upper member of FIG. 2 along the lines IV-IV of FIG. 4E;

FIG. 5 is a top-down exploded view of a base of the assembly of FIG. 1;

FIG. 6 is a bottom-up exploded view of the base of FIG. 5;

FIG. 7A is a top-down perspective view of the base of FIG. 5;

FIG. 7B is a bottom-up perspective view of the base of FIG. 5;

FIG. 7C is a side view of the base of FIG. 5;

FIG. 7D is a top view of the base of FIG. 5;

FIG. 7E is a bottom view of the base of FIG. 5;

FIG. 7F is a cross-section view of the base of FIG. 5 along the lines VII-VII of FIG. 7E;

FIG. 8 is a top-down perspective view of the assembly of FIG. 1, in an open configuration;

FIG. 9 is a bottom-up perspective view of the assembly of FIG. 1, in the open configuration;

FIG. 10 is a cross-section view of the assembly of FIG. 1;

FIGS. 11A-11C are perspective views of the assembly of FIG. 1 in different operational positions;

FIG. 12 is a bottom-up perspective view of a second example embodiment of an herb holding assembly for use in a vaporizer;

FIG. 13 is a top-down exploded view of an upper member of the assembly of FIG. 12;

FIG. 14 is a bottom-up exploded view of the upper member of FIG. 13;

FIG. 15 is a top-down perspective view of the upper member of FIG. 13;

FIG. 16 is a top-down exploded view of a base of the assembly of FIG. 12;

FIG. 17 is a bottom-up exploded view of the base of FIG. 16; and

FIG. 18 is a top-down perspective view of the base of FIG. 16.

### DETAILED DESCRIPTION

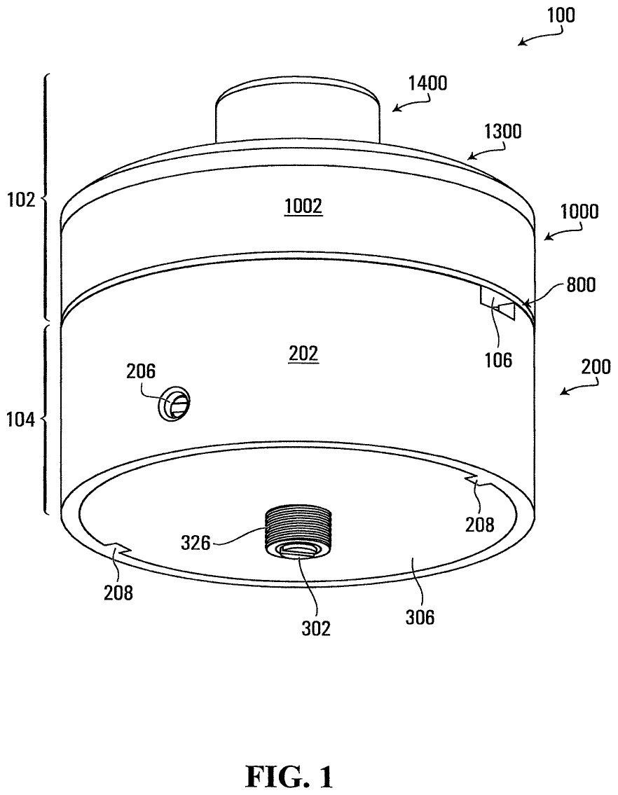

Referencing FIG. 1, assembly 100 has an upper member 102 magnetically joined to a base 104.

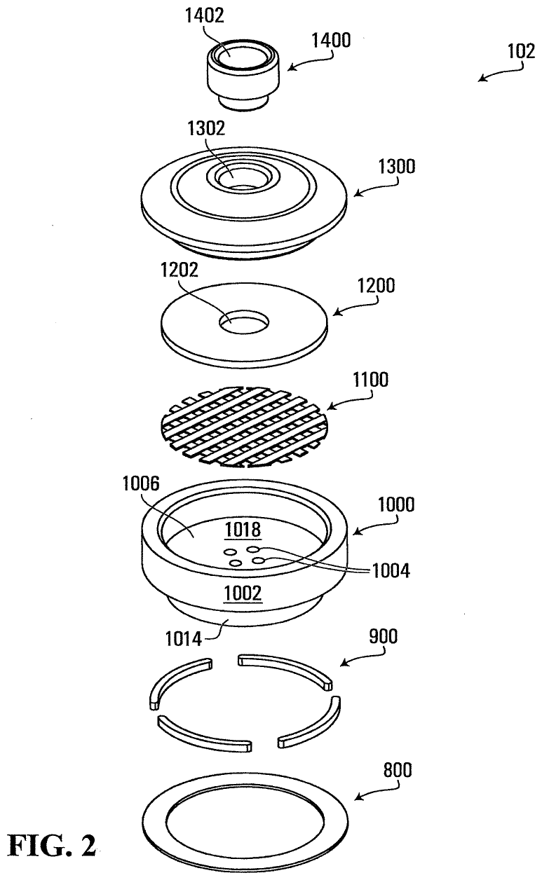

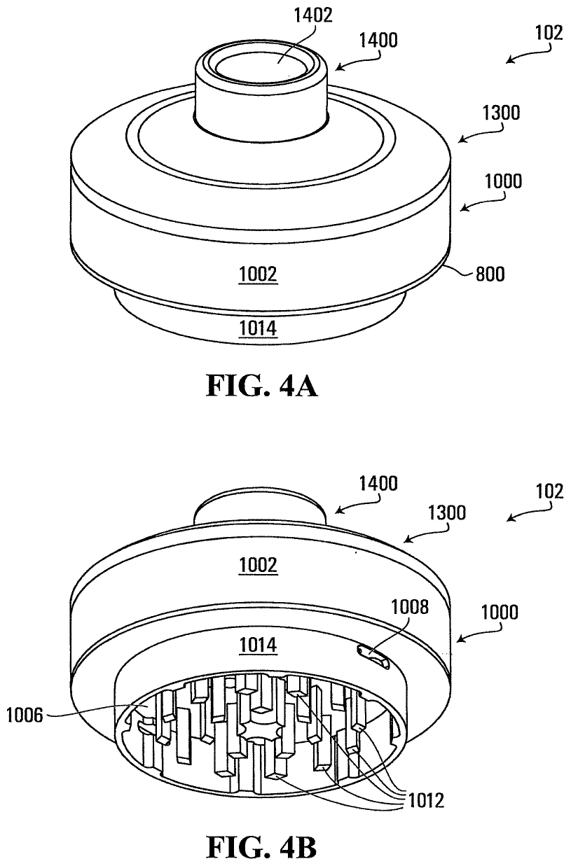

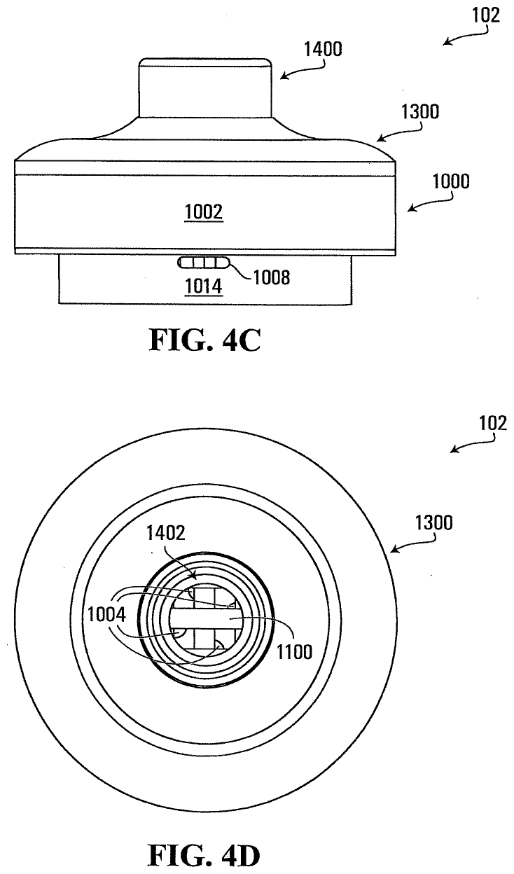

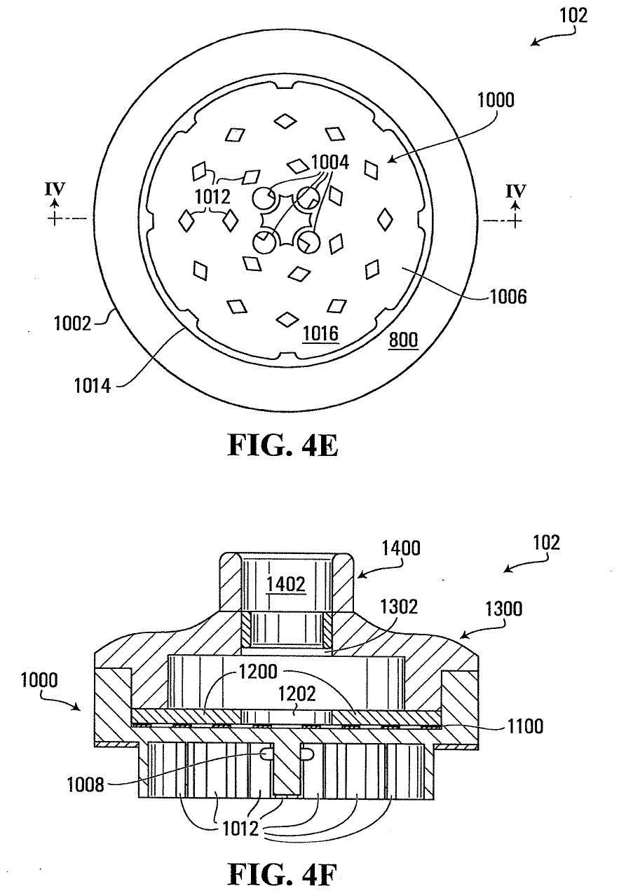

Turning to FIGS. 2 to 4, upper member 102 has a mouthpiece 1400 press fit to a top cap 1300. The top cap 1300 is press fit or screwed to a top grinding element 1000 and sandwiches a thermal insulator 1200 and filter 1100 between the top cap and a top face 1018 of a disc-shaped wall 1006 of the top grinding element 1000.

Filter 1100 may be a mesh filter, a foam filter, a carbon filter, a charcoal filter, a film filter, or a self-contained water filter amongst others.

Mouthpiece 1400, top cap 1300, and insulator 1200 have openings 1402, 1302, and 1202 respectively, to allow air to flow from top grinding element 1000, through filter 1100 to mouthpiece 1400.

Disc-shaped wall 1006 also has axially directed openings 1004 to allow air from below wall 1006 to flow from top grinding element 1000 to mouthpiece opening 1402.

Teeth 1012 project downwardly from the bottom face 1016 of the disc-shaped wall 1006 of top grinding element 1000.

The disc-shaped wall 1006 of top grinding element 1000 forms a floor for an upper annular wall 1002 of the top grinding element. A smaller diameter lower annular wall 1014 depends from the disc-shaped wall.

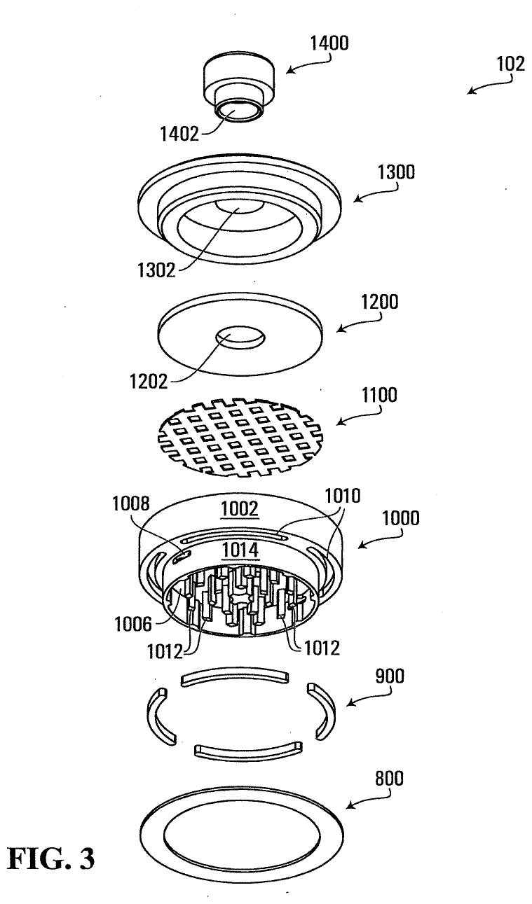

Arcuate grooves 1010 (FIG. 3) extend into the bottom surface of the disc-shaped wall 1006 between the upper and lower annular walls 1002, 1014. These grooves receive arcuate magnets 900 which, as will become apparent, function to releasably secure upper member 102 to base 104. An O-ring 800 is received on lower annular wall 1014 and covers magnets 900.

Lower annular wall 1014 has one or more radial passageways 1008 (FIG. 3) therein to allow air to flow into top grinding element 1000.

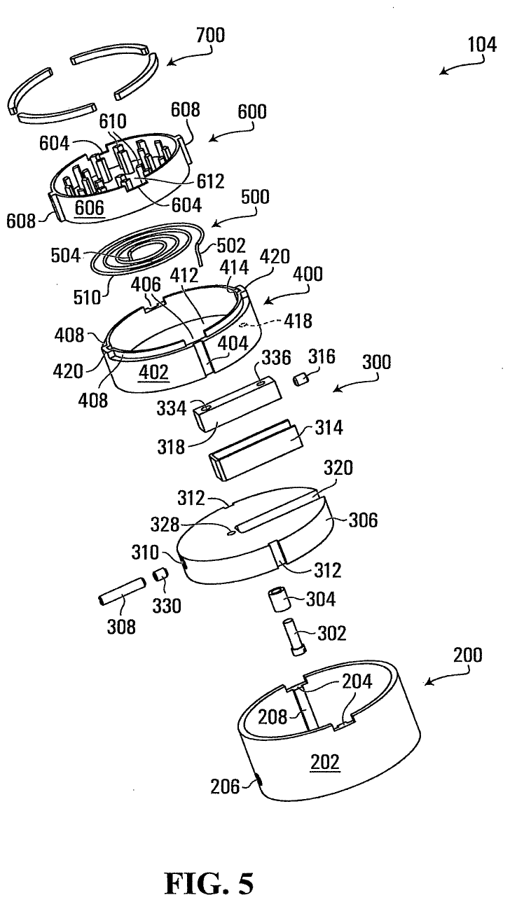

Turning to FIGS. 5-7, base 104 has a casing in the nature of a cylindrical sleeve 200 with upper notches 204 that act as sleeve air passageways, a radially directed bore 206, and a pair of opposed axially extending keys 208 extending from the base of notches 204.

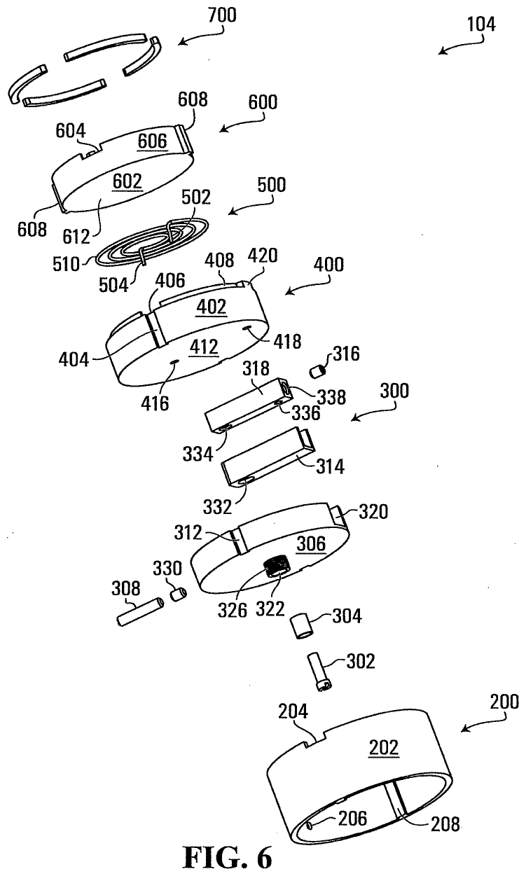

A terminal assembly 300 has a disc-shaped negative terminal block 306 with an opening 322 (FIG. 6) extending along its central axis. Opening 322 is partially defined by a downwardly directed and externally threaded central annular projection 326. Block 306 also has a radially directed notch 320 that extends past the central axis of the block and also opens to the top of the block. An axial opening 328 (FIG. 5) extends into the top of the block and a radially directed bore 310 (FIG. 5) extends from a side of the block to axial opening 328. Block 306 also has a pair of opposed axially extending keyways 312. The negative terminal block sits within sleeve 200 with keyways 312 receiving keys 208 of the sleeve 200 thereby locking the block 306 against rotation.

A pin 330 is inserted into radial bore 310 of the negative terminal block 306 and pushed into position by a set screw 308. As will become apparent hereinafter, this allows the pin 330 to hold a negative terminal of a heater against an inner wall of the negative terminal block 306. Set screw 308 is positioned to lie within sleeve bore 206 and negative terminal block bore 310 in order to hold the negative terminal block within the sleeve.

As best shown in FIG. 6, a rectangular insulative sleeve 314 is received by groove 320 of the negative terminal block 306 and the insulative sleeve 314 receives, in turn, rectangular positive terminal block 318. The inner end of the insulative sleeve has an axially directed opening 332 aligned with the central opening 322 of the negative terminal block. The positive terminal block 318 has a smaller diameter axially directed opening 334 also aligned with the central opening 322 of the negative terminal block 306. An insulative ring 304 projects through central opening 322 of block 306 and opening 332 of insulative sleeve 314 to abut the bottom of positive terminal block 318. A conductive, positive terminal pin 302 projects through openings 322, 332, and 334 to connect to the positive terminal block 318.

The outer end of the positive terminal block 318 has an axially directed unthreaded opening 336 extending from its top to bottom surfaces and a radially directed threaded opening 338 extending from its outer end to the unthreaded opening 336. A conductive set screw 316 is threaded into threaded opening 338 in order to hold a positive terminal of the heater to the positive terminal block 318, as will become apparent hereinafter.

Positive terminal pin 302 and negative terminal annular projection 326 protrude from an end of sleeve 200 and act as a connector to allow heater 500 to couple to with a source of electric power. In particular, annular projection 326 may have external threading that accords with any one of a variety of standard connectors used in the vaporizer industry, such as the 510 connector, amongst others. To accord to with the industry standard 510 connector, annular projection 326 may have an external diameter of 7 mm, may project outwardly by 3 to 5 mm from negative terminal block 306, and may have one to 10 threads with a thread pitch of 0.5 mm.

An insulator 400 has an annular wall 402 and a floor 412. The annular wall 402 has upper notches 406 that act as air passageways, a pair of opposed outwardly directed keyways 404, a pair of opposed inwardly directed keyways 414 and upper arcuate ledges 408. The floor 412 has axial openings 416 and 418 (FIG. 6). The insulator 400 sits inside sleeve 200 in abutment with the negative terminal block 306 with its outwardly directed keyways 404 received by keys 208 of the sleeve 200 thereby locking the insulator 400 against rotation within the sleeve. In this position, the upper notches 406 of the insulator are aligned with the upper notches 204 of the sleeve 200.

A resistive coil 510 of a heater 500 sits on the floor 412 of insulator 400. A finger 502 at an outer end of the coil 510 projects through opening 418 in floor 412 and is received by opening 336 in the positive terminal block 318 and held to this block by set screw 316. This finger acts as the positive terminal for the heater 500. A second finger 504 at an inner end of the coil 510 extends through opening 416 of floor 412 and is received by opening 328 (FIG. 5) of the negative terminal block 306 and held to the negative terminal block by conductive pin 330 that is pushed against finger 504 by set screw 308. This finger acts as the negative terminal for the heater 500.

The upper arcuate ledges 408 of the insulator 400 receive arcuate magnets 700 that, with the insulator sitting inside sleeve 200 are sandwiched between ledges 408 of the insulator 400 and sleeve 200. The magnets are held against sliding by abutments 420 of the insulator 400.

A bottom grinding element 600 has an annular wall 606 and a floor 612. The annular wall 606 has upper notches 604 that act as air passageways and a pair of opposed keys 608. The floor 612 supports upwardly directed teeth 610. The bottom grinding element 600 sits within insulator 400 with its keys 608 received by inwardly directed keyways 414 of the insulator 400. This locks the bottom grinding element 600 against rotation within the insulator 400 with the upper notches 604 of the bottom grinding element 600 aligned with upper notches 406 of the insulator 400 and notches 204 of the sleeve 200.

With the bottom grinding element 600 sitting within the insulator 400, heater 500 is sandwiched between the floor 612 of the bottom grinding element 600 and the floor 412 of insulator 400.

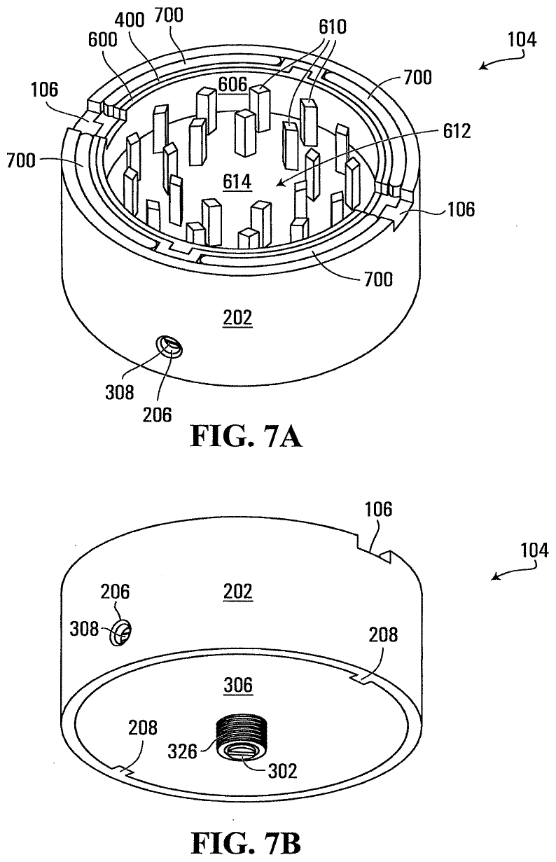

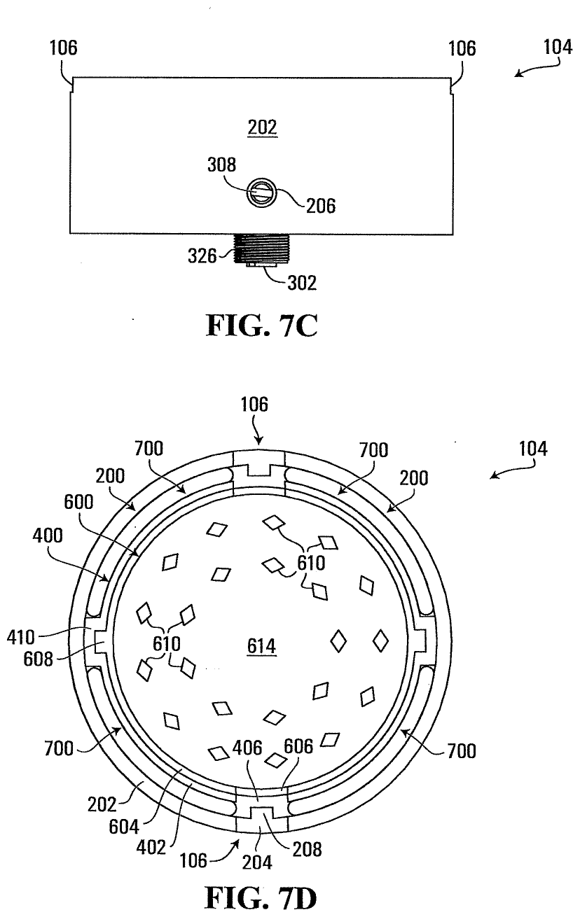

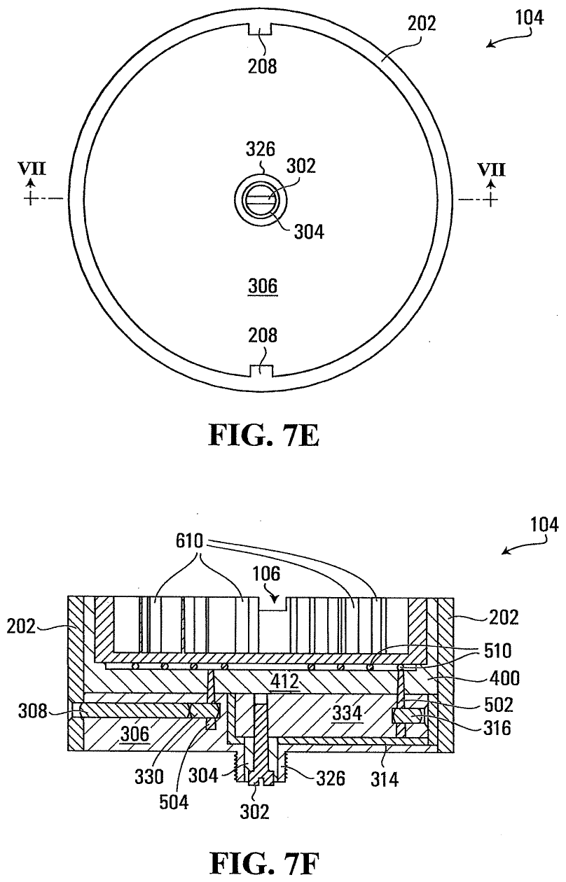

It will be apparent that once bottom grinding element 600 and insulator 400 are secured within sleeve 200 the bottom grinding element air notches 604, insulator air notches 406, and sleeve notches 204 (collectively, base notches 106) are aligned and in fluid communication with one another (FIG. 7).

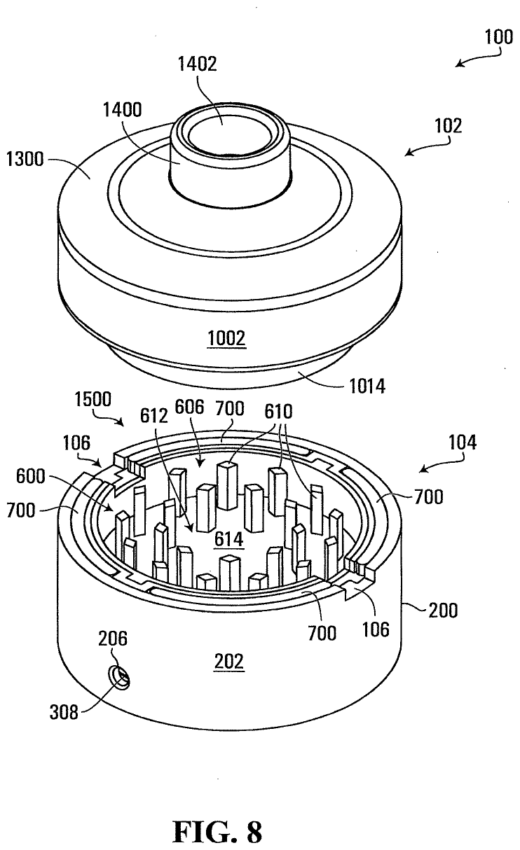

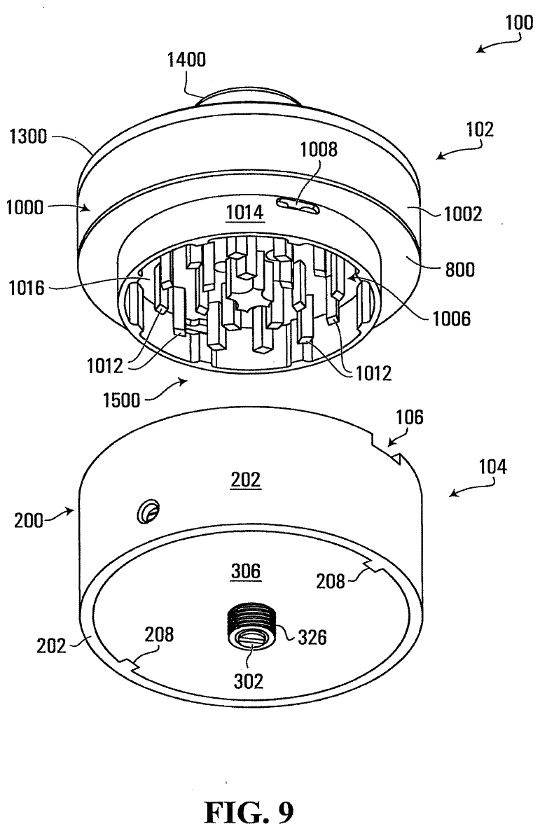

FIGS. 8-9 show the assembled upper member 102 and assembled base 104 separated from each other. From this it will be apparent teeth 1012 of upper member 102 and teeth 610 of base 104 project toward one another. When upper member 102 and base 104 are brought together, a bottom face 1016 of disc-shaped wall 1006 of top grinding element 1000 opposes a top face 614 of floor 612 of bottom grinding element 600 and bottom annular wall 1014 of the top grinding element 1000 partially overlaps with annular wall 606 of the bottom grinding element 600. Top grinding element 1000, along with bottom grinding element 600, thereby define a grinding chamber 1500. When upper member 102 and base 104 are brought together, O-ring 800 creates a seal between the upper member and base.

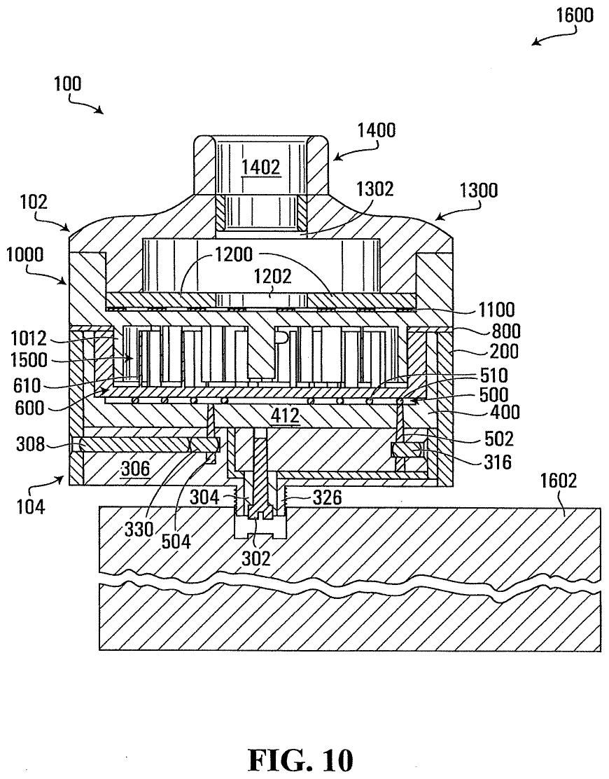

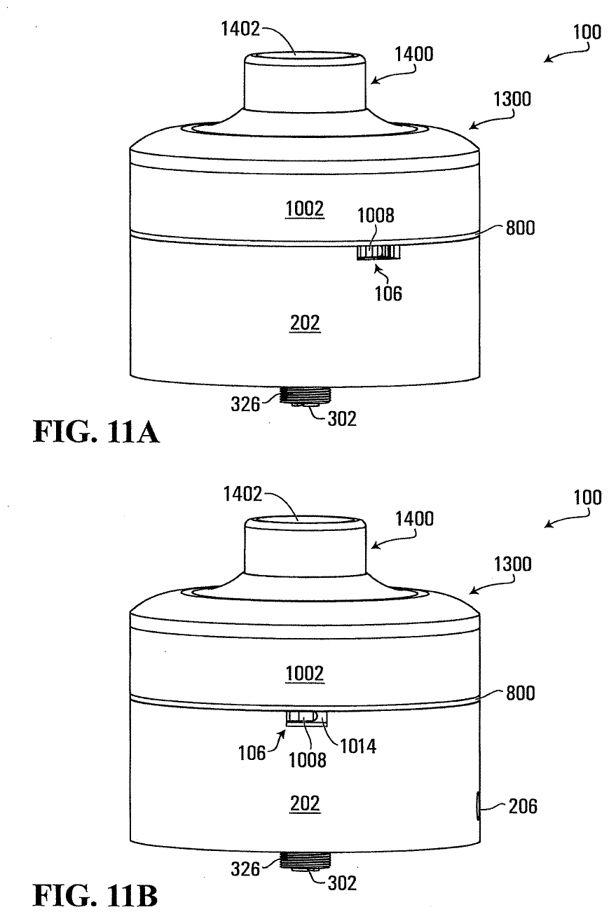

Turning to FIG. 10, assembly 100 can be attached to a control unit 1602 to form a vaporizer 1600. More specifically, control unit 1602 has a threaded connector (such as a 510 connector) suitable for receiving the externally threaded annular projection 326 of base 104. Coupling base 104 and control unit 1602 causes annular projection 326 and protruding pin 302 to couple with negative and positive terminals, respectively, of a battery unit (not shown) within control unit 1602. Annular projection 326 and pin 302 thereby provide an electric connection between the battery unit of control unit 1602 and heater 500.

With the assembly 100 connected to the control unit, electricity may be selectively supplied through pin 302 and annular projection 326 to heater 500.

Control unit 1602 may include a controller (e.g. a microprocessor or a control circuit, not shown) to control the power output to heater 500, and thereby control the heat generated by heater 500. Control unit 1602 may also include one or more input devices to receive a user input indicative of a power output and may set the power output based on the user input.

In use, prior to bringing the top member 102 and base 104 together, the bottom grinding element 600 may be filled with one or more herbs or other plant material. When brought together, grinding chamber 1500 is formed and the attraction of magnets 900 of the upper member 102 to magnets 700 of the base 104 releasably hold the upper member 102 and base 104 together.

Heat may then be applied to grinding chamber 1500 by controlling control unit 1602 to provide electric power to heater 500. Heater 500 is operatively associated with grinding chamber 1500 and heats the contents of grinding chamber 1500 indirectly. More specifically, heater 500 abuts bottom face 602 of floor 612, which is external to grinding chamber 1500. The contents of grinding chamber sit atop floor 612 and do not contact this bottom face 602. Thus, the contents of grinding chamber 1500 are prevented from directly contacting heater 500. Avoiding direct contact with heater 500 reduces the risk of the contents of the grinding chamber 1500 burning. Floor 612, instead, spreads the heat from heater 500 evenly across its surface and to annular wall 606 and teeth 610, thereby reducing this risk.

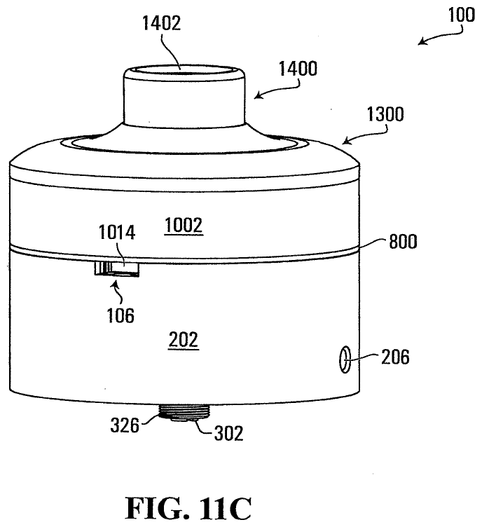

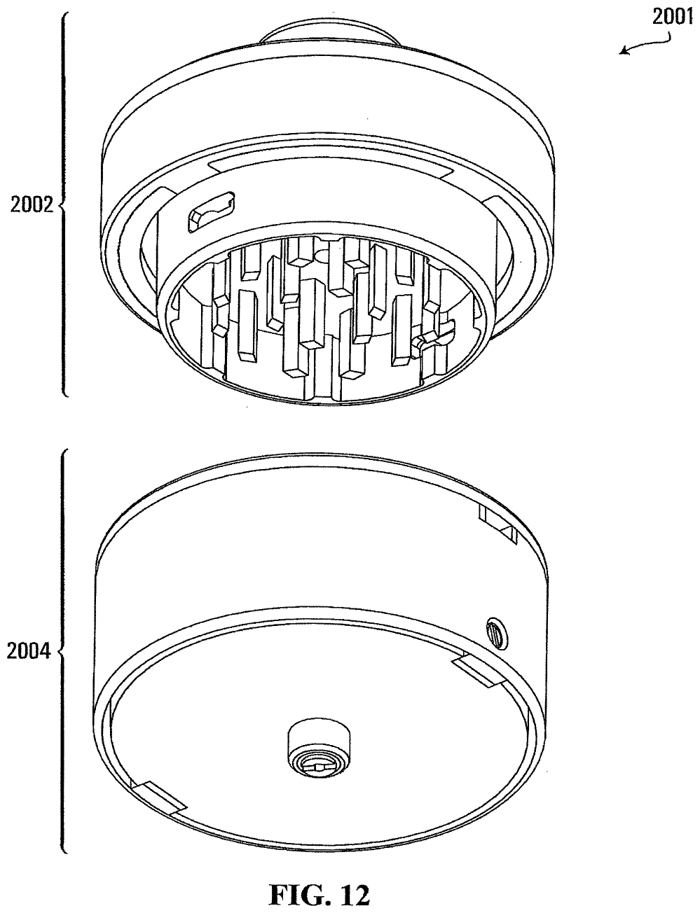

Heating grinding chamber 1500 cause constituents of herbs held in the chamber to vaporize. The temperature of grinding chamber 1500 may be controlled by control unit 1602. The temperature of grinding chamber 1500 may also be controlled by adjusting the alignment of base vents 106 and radial passageways 1008. In particular, base vents 106 and passageways 1008 may be brought into alignment by selectively rotating top grinding element 1000 relative to bottom grinding element 600, thereby opening an airway to grinding chamber 1500. The base vents 106 and passageways 1008 may be fully aligned (FIG. 11A), partially aligned (FIG. 11B), or fully out of alignment (FIG. 11C). When the base vents 106 and passageways 1008 are fully or partially aligned, air will flow into chamber 1500 and out of the mouthpiece opening 1402, thereby reducing the temperature within the chamber.

The herb or other material within grinding chamber 1500 may be heated unevenly, with portions of the herb abutting top face 614 of floor 612 heated more quickly and at a higher temperature than portions of the herb further away from top face 614. To avoid this, the contents of grinding chamber 1500 may be agitated and ground by rotating upper member 102 of assembly 100 relative to the base 104 which will bring new portions of the herb into contact with top face 614 of floor 612 and with teeth 610, thereby providing more even heating.

With the airway to the grinding chamber 1500 open and the herb, or other material, at a desired temperature, a user may inhale through mouthpiece 1400, thereby causing air to flow through the base airway 106 and radial passageways 1008 of the top grinding element 1000 into the top grinding element, then through the axial openings 1004 of top grinding element 1000, through air filter 1100 and through mouthpiece opening 1402.

It will be apparent from the foregoing that top grinding element 1000 may be selectively rotated relative to bottom grinding element 600 to open an airway to grinding chamber 1500 and may also be rotated to agitate the herb, or other material, within grinding chamber 1500. Thus, rotation of the top grinding element relative to the bottom grinding element serves two purposes. This double use reduces the number of parts needed and therefore reduces manufacturing cost. This dual use also reduces the complexity of the user interface, and may be more convenient to the user.

While heater 500 has been described as a coil resistive heater, any other suitable heater such as a conduction, convection, radiation, or ultrasonic heater could be used. Further, while heater 500 is shown as spiral shaped, it may square-shaped, rectangle-shaped, or circle-shaped, and may be formed of a mesh or a series of links.

Further, floor 612 of the bottom grinding element 600 may have grooves (not shown) in which the heater 500 may be embedded. Similarly, floor 412 of the insulator 400 may also have grooves (not shown) in which the heater may also be embedded. By embedding the heater 500 in grooves of the floor 612 and in grooves of the floor 412, the airspace between the bottom grinding element 600 and the top of the insulator 400 is minimized. This improves efficiency of the heater as it minimizes the wasted heating of air in this airspace. Furthermore, with the heater 500 embedded in grooves of the floor 612, the heater 500 is in contact with a larger surface area of the bottom grinding element 600, which may increase the efficiency of heater 500 in heating the chamber 1500. With the heater 500 is embedded in grooves of the floor 412, the heater 500 is in contact with a larger surface area of the insulator 400, which may increase the efficiency of the insulator 400 at insulating the heat output of the heater 500. In addition, by embedding the heater 500 in grooves of either the floor 612 or the insulator 400, base 104 may occupy a smaller volume.

In an alternative embodiment, vaporizer 1600 may have a base assembly that is permanently mounted to a control unit.

In the illustrated embodiment, the insulator is positioned above the filter but in an alternate embodiment, insulator 1200 is positioned below filter 1100.

In an alternative embodiment, notches that form base notches 106 (i.e. bottom grinding element notches 604, insulator notches 406, and notches 204) may be provided in the form of through holes instead of notches. Further, although the figures show two of each notches 604, 406, 204, a different number of notches may be provided.

In an alternative embodiment, axially directed openings 1004 of top grinding member 1000 may be selectively opened and closed by rotating any one of filter 1100, insulator 1200, or top cap 1300.

In one embodiment, radial bore 310 of negative terminal block 306 may be threaded and configured to receive a threaded pin 330 and set screw 308. In another embodiment, radial bore 310 may be unthreaded. An unthreaded pin 330 and set screw 308 may be press-fit into the unthreaded radial bore 310. Both pin 330 and set screw 308 may be made of either a conductive or non-conductive material.

In one embodiment, chamber 1500 is heated to a temperature in the range of 100 to 240° C.

In one embodiment, casing 200 and top grinding element 1000 may have visual indicator that the base vents 106 and radial passageways 1008 are in alignment. The visual indicator could be, for example, a marking or an indentation.

While the described casing 200 for the base 104 is in the nature of a sleeve, other types of casings may be used, such as a casing that envelopes the sides and bottom of the base 104 other than annular projection 326 and pin connector 302.

Teeth 1012 of top grinding element 1000 and teeth 610 of bottom grinding element 600 may have any number of shapes and sizes, such as square-shaped, s-shaped, x-shaped, diamond-shaped, star-shaped. In addition, each tooth of each grinding element 1000, 600 may be shaped differently from other teeth of that grinding element 1000, 600 to facilitate improved grinding and agitation of the herbs in chamber 1500.

As will be appreciated, a wide variety of materials may be used to construct various elements of assembly 100. For example, the top and bottom grinding elements 1000, 600 may be made using materials suitable for grinding an herb, such metallic materials (e.g. stainless steel or aluminum), ceramics, or glass. Insulators 1200, 400 may be made using thermally insulating materials that can withstand the high temperatures needed for vaporizing herbs, such as polymers, plastics, wood, glass, or composites. Casing 200 may be made of any number of materials, such as metals, woods, composites, ceramics, glass, polymers, or plastics.

Turning to FIG. 12, which illustrates a second example embodiment of an assembly for vaporizing an herb, wherein like parts to those of the assembly 100 of FIGS. 1 to 11 are given like reference numerals, assembly 2001 has an upper member 2002 magnetically joined to a base 2004. For operation, the assembly 2001 is joined to a control unit, such as control unit 1602 (FIG. 10).

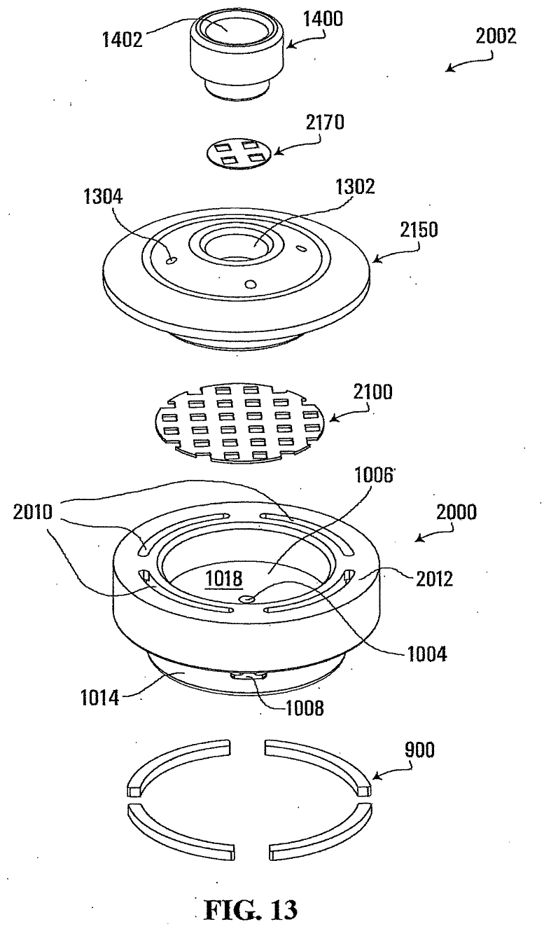

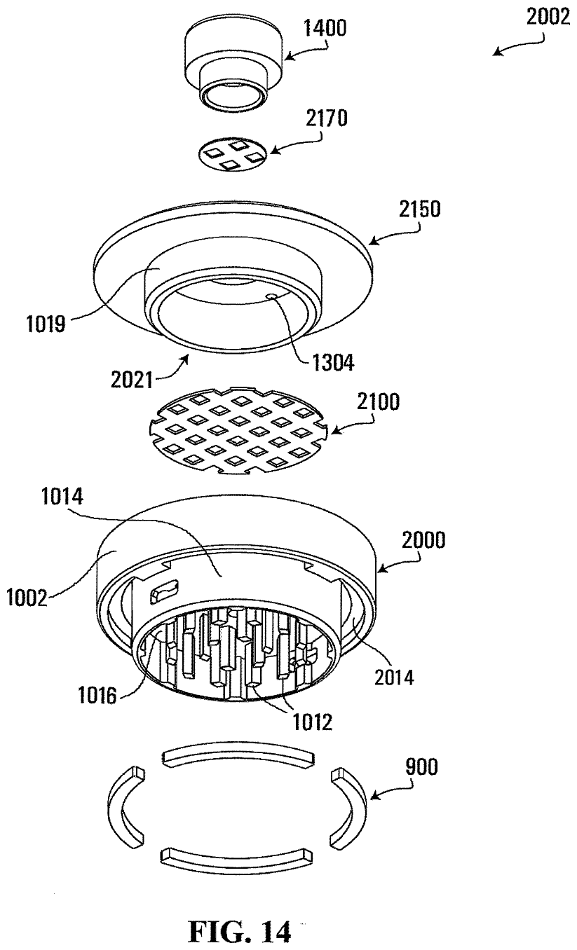

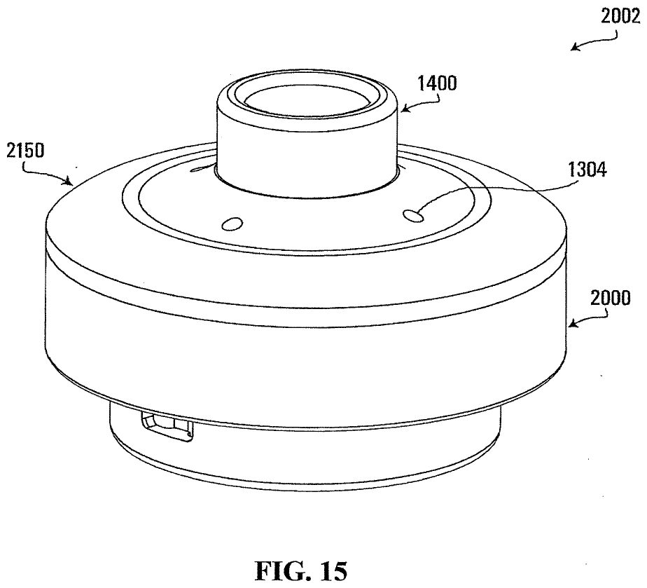

Turning to FIGS. 13 to 15 which illustrate the upper member 2002, mouthpiece 1400 is press fit or otherwise attached within opening 1302 of cap 2150 after a filter 2170 is placed within the cap. The cap 2150 differs from cap 1300 in the inclusion of axial through holes 1304 that are inbound of the annular wall 1019 of the cap. As with the first embodiment, the cap is attached to a top grinding element 2000 to sandwich a filter 2100 between its annular wall 1019 and the top face 1018 of ceiling wall 1006 of the top grinding element 2000. With the cap 2150 attached to the top grinding element 2000, an air-vapor mixing chamber 2021 is defined by the annular wall 1019 of the cap and the grinding element top face 1018. As the user draws from mouth piece 1400, vapor is introduced into mixing chamber 2021 through axial through hole 1004 from the grinding chamber. Ambient air is also introduced into the air-vapor mixing chamber through holes 1304 and the combined air-vapor mixture in 2021 exits through 1402 of mouth piece 1400. This air-vapor mixing action has a cooling effect on the vapor introduced from the grinding chamber through hole 1004. Filters 2100 and 2170 could be of mesh, foam, cotton, composite, or any other material that may contain various flavors or flavor emitting properties as air passes through, binding to or mixing with the air-vapor mixture that passes through it. Alternatively, filters 2100 and/or 2170 could have odor absorbing properties, filtering out the smell of the air-vapor mixture from the grinding chamber.

Top grinding element 2000 differs from top grinding element 1000 in it has arcuate slots 2010 extending axially through the lip 2012 of the grinding element which provide cooling and insulation between the top grinding chamber annular wall 1014 and the external body annular wall 1002. The concentric arcuate magnets 900 in arcuate grooves 2014 are also separated with a circular air insulation gap, concentrically expanding from annular wall 1014 towards inner face of annular wall 1002. This prevents direct conduction heat from 1014 to reach 1002 and arcuate magnets 900 in arcuate grooves 2014. As with grinding element 1000, slots 1010 in the bottom of lip 2012 receive magnets 900 such that the magnets are aligned end-to-end so as to form a circular pattern.

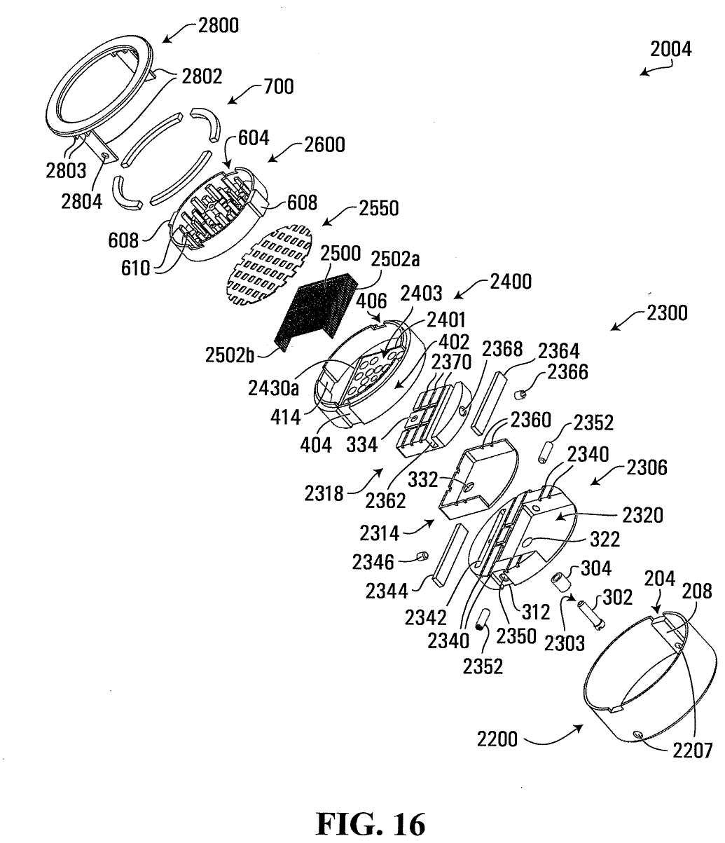

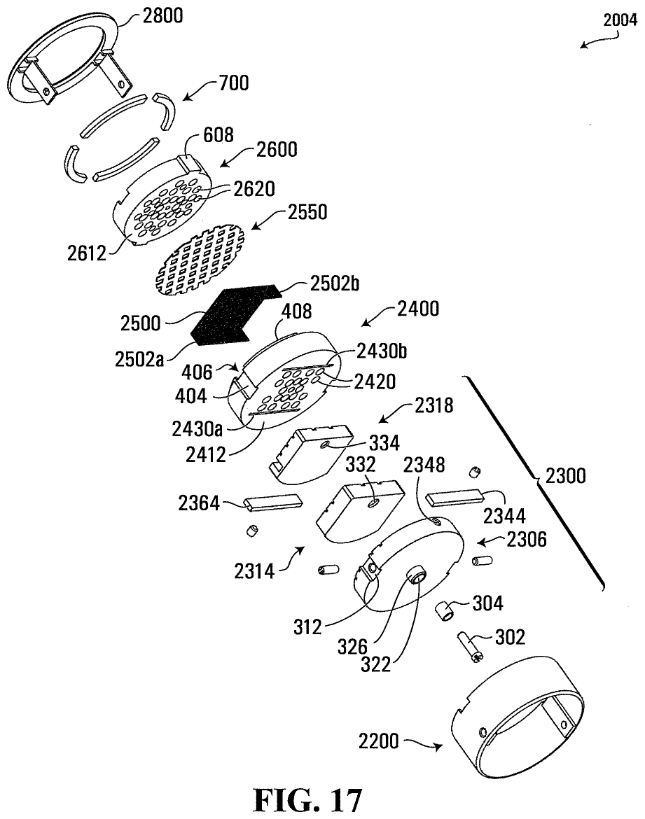

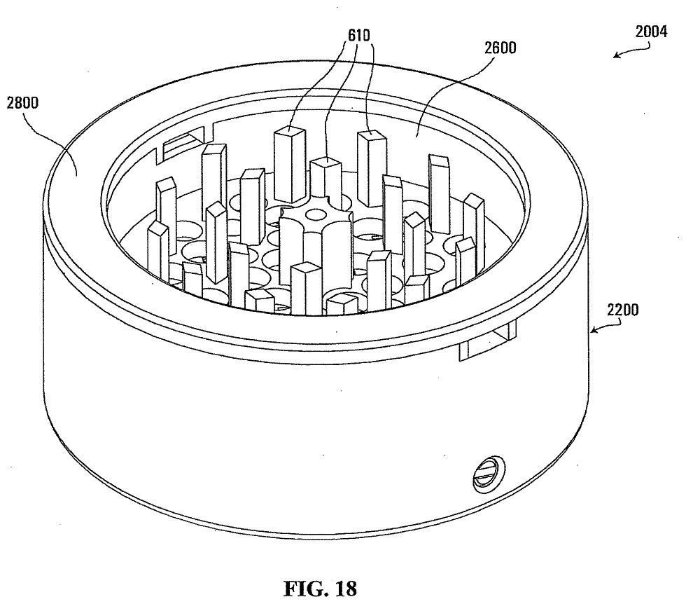

Turning to FIGS. 16 to 18 which illustrate the base 2004, sleeve 2200 differs from sleeve 200 in the inclusion of bores 2207 through the wall of the sleeve at keys 208.

With terminal assembly 2300, disk-shaped negative terminal block 2306 differs from negative terminal block 306 in a number of respects. Firstly, radially directed groove 2320 of negative terminal block 2306, which extends past the central axis of the block, and hence past central opening 322, is enlarged and a plurality of criss-crossed transverse notches 2340 extend into the top of the negative terminal block. The top of the block 2306 also has a slot 2342 receiving a bar 2344. A set screw 2346 is received within a threaded bore 2348 in block 2306 to press against the bar 2344. The keyways 312 of block 2306 have threaded bores 2350 which receive set screws 2352.

Electrically insulative sleeve 2314 is received by groove 2320 of the negative terminal block and the insulative sleeve 2314 receives positive terminal block 2318. The top of the wall of the insulative sleeve 2314 has notches 2360 that align with notches 2340 of the negative terminal block 2306. The positive terminal block 2318 has a slot 2362 which receives a bar 2364. A set screw 2366 is threaded into threaded bore 2368 and presses against bar 2364. Criss-crossed transverse notches 2370 extend into the top of the positive terminal block 2318. Notches 2370 are aligned with both the transverse notches 2360 of the insulative sleeve 2314 and the transverse notches 2340 of the negative terminal block. Heat and electrical insulator 2400 has a square central depression 2401 partially bounded by a pair of parallel through slots 2430a, 2430b. The floor 2412 of insulator 2400 within the perimeter of this depression is perforated with circular openings 2420.

A mesh heater 2500 has legs 2502a, 2502b that extend through slots 2430a, 2430b of insulator 2400 respectively. Leg 2502a further extends into slot 2362 of positive terminal block 2318 and may be clamped in place between the inner side wall of the slot 2362 and bar 2364 by turning set screw 2366 to urge the bar 2364 against leg 2502a. Similarly, leg 2502b further extends into slot 2342 of the negative terminal block 2306 and may be clamped in place between the inner side wall of the slot 2342 and bar 2344 by turning set screw 2346 to urge bar 2344 against leg 2502b.

A filter 2550 sits within insulator 2400 atop mesh heater 2500. This filter prevents ground herb in bottom grinding element 2600 from falling through openings 2420 in the floor of element 2400 and prevent direct contact with heater 2500.

Bottom grinding element 2600 differs from grinding element 600 in the inclusion of the openings 2620 in its floor 2612.

As in the first embodiment, arcuate magnets 700 sit on the periphery of insulator 2400, between arcuate ledges 408 of the insulator 2400 and sleeve 2200 such that the magnets are aligned end-to-end.

The base 2004 may be assembled and held together as follows. The terminal assembly 2300 is assembled by nesting the positive terminal block 2318 within electrically insulative sleeve 2314 and the insulative sleeve 2314 within negative terminal block 2306. The heat/electrical insulator 2400 is then placed atop the terminal assembly 2300 with its slots 2430a, 2430b aligned with slots 2362 and 2342, respectively. This also aligns keyways 404 of insulator 2400 with keyways 312 of negative terminal block 2306 of terminal assembly 2300. The legs 2502a, 2502b of heater 2500 are then inserted through respective slots 2430a, 2430b of the heat/electrical insulator 2400 and into respective slots 2362 and 2342 of the terminals. Set screws 2366 and 2346 are then turned to clamp the legs of the heater between respective bars 2364 and 2344 and the inner walls of respective slots 2362, 2342. The terminal assembly with the insulator 2400 and heater 2500 is then slid into sleeve 2200, with the keys 208 of the sleeve receiving keyways 312 and 404 of the negative terminal block 2306 and insulator 2400, respectively. Next the filter 2550 is placed within the insulator 2400 and bottom grinding element 2600 inserted into insulator 2400 with the keys 608 of the grinding element received by the keyways 414 of the insulator 2400. Because of the square depression 2401 in the floor of the insulator 2400, there will be an air chamber 2403 formed between the insulator 2400 and bottom grinding element 2600. With the bottom grinding element seated, the arcuate magnets 700 are inserted into the sleeve 2200 so as to be disposed between the sleeve and the arcuate ledges 408 of the insulator 2400. Next, the ears 2802 of a retaining ring 2800 are inserted into the sleeve 2200 between the keys 208 of the sleeve and the respective keyways 312, 404 of the negative terminal block 2306 and the insulator 2400. With the retaining ring in place, detents 2803 at the base of each ear 2802 of the retaining ring prevents sliding movement of each magnet 700 in one direction and the keys 608 of the bottom grinding element block sliding movement of the magnets 700 in the opposite direction. Lastly, screws 2352 are threaded into threaded openings 2350 in the negative terminal block 2306 through openings 2207 in sleeve 2200 and openings 2804 in the ears 2802 of the retaining ring 2800.

To complete assembly of the bottom assembly 2004, insulative ring 304 is press fit into opening 322 of annular projection 326 of the negative terminal block 2306 and pin 302 is inserted through the insulative ring 304 and through 332 of insulative sleeve 2314 and is press fit into opening 334 of the positive terminal block 2318. Alternatively, the pin 302 and opening 334 may be threaded so that the pin is threaded into opening 334.)

With the top assembly 2002 magnetically joined to the base 2004, the retaining ring 2800 (similarly to O-ring 800 of the first embodiment) seals the two together. Further, the top of retaining ring 2800 abuts the bottom of lip 2012 of the top grinding element 2000.

With the top assembly 2002 magnetically joined to the base 2004, there is an axial flowpath through the assembly 2001. More specifically, the negative terminal block 2306 is sized so that with the base 2004 assembled, there is an annular gap between the sleeve 2200 and the periphery of the negative terminal block. Therefore, with a user drawing on mouthpiece 1400, air flows from the bottom of the (open ended) sleeve 2200 through the annular gap between the sleeve and negative terminal block to the aligned transverse notches 2340, 2360, 2370 of the negative terminal block, insulative sleeve and positive terminal block. These aligned transverse notches allow the air to pass under the floor 2412 of heat/electrical insulator 2400 where the air may flow through openings 2420 in this floor into air chamber 2403 where the air passes through mesh heater 2500. The air then passes through filter 2550 and then through openings 2620 in the floor 2612 of the bottom grinding element 2600 so as to reach the grinding chamber defined by the bottom and top grinding elements 2600, 2000. Air then flows through opening 1004 in the ceiling wall 1006 of the top grinding element and through filter 2100 into mixing chamber 2021. From the mixing chamber, air flows through filter 2170 and mouthpiece opening 1402, mixed with ambient air drawn through openings 1304 of cap 2150 due to the vacuum created in the mixing chamber by the user pulling air through mouthpiece opening 1402. The ambient air from openings 1304 acts to cool vapors drawn through hole 1004.

In operation, the assembly 2001 is joined to a control unit, such as control unit 1602 (FIG. 10) and, as with the first embodiment, material in the grinding chamber may be ground and agitated by rotating the top grinding element with respect to the bottom grinding element.

In consequence of the axial airflow path, even if the top grinding element 2000 is rotated so as to close the radial flowpath into assembly 2001, air is free to flow axially through the assembly 2001.

The airflow path through the aligned notches 2340, 2360, 2370 is more constrained than the airflow path through circular openings 2420 in the floor of insulator 2400. Consequently, the airflow slows as the air passes through openings 2420 since the surface area of the airflow path increases. Also, the airflow path through the openings 2420 is more constrained than through the chamber 2403 defined between the insulator 2400 and the bottom grinding element 2600 such that this chamber acts as an air expansion chamber, further slowing the airflow speed and increasing the surface area of the airflow path. The floor of the bottom grinding element has a greater number of openings than that of the floor of the insulator 2400 so as to maintain this lower airflow speed. This reduction in the speed of the airflow, and the increased surface area of the airflow path, allows more time for the air to be heated by the heater 2500 so that the air, being hotter, will more effectively vaporize material in the grinding chamber.

As assembly 2001 is designed to operate with a solid material in the grinding chamber, the assembly runs hotter than an assembly designed for use with juice. The provision of annular airflow between sleeve 2200 and the negative terminal block 2306 and insulator 2400 helps keep the sleeve cool to the touch of the user despite this hotter operation. Also, the through arcuate slots 2010 in the top grinding element 2000 assist in keeping the top grinding element cool to the touch.

Pin 302 has a small central bore 2303. This pin terminates at hole 334 or may extend into one of the openings of 2420. One of the openings 2420 in the floor of insulator 2400 lines up with opening 334. Given this arrangement, the control unit 1602 (FIG. 10) attached to pin 302 can be designed to accommodate a juice that the user can squirt through the central bore of the pin and through an opening in the floor of the insulator 2400 so that the juice reaches the heater 2500. The heater will therefore vaporize this juice. Thus, the juice can be used to flavor vapors produced by the assembly 2001.

In the described second embodiment, the user has the option of increasing airflow through the device by turning the top grinding element to open a radial flowpath. This can assist in controlling the heat in the grinding chamber and in clearing remaining vapor in the grinding chamber, but it is not necessary for operation. Therefore, the assembly of the second embodiment may be modified so that no radial airflow path is created with rotation of the top grinding element.

Openings 1304 in cap 2150 mix some fresh air with the vapor generated in the grinding chamber when a user draws on the mouthpiece 1400. While this may be desirable, in a modification, these openings 1304 are omitted.

Of course, the above described embodiments of the present disclosure are intended to be illustrative only and in no way limiting. The described embodiments are susceptible to many modifications of form, arrangement of parts, details and order of operation. The invention, rather, is intended to encompass all such modification within its scope, as defined by the claims.

What is claimed is:

1. An assembly for use in an herb vaporizer, comprising:
a plurality of elements defining an air passageway with an inlet section, a heater chamber, a grinding chamber, and an outlet section;
said inlet section in fluid communication with said heater chamber;
an outlet of said heater chamber in fluid communication with said grinding chamber;
an outlet of said grinding chamber in fluid communication with said outlet section;
a heater disposed within said heater chamber, said heater configured to allow passage of air therethrough; and
a mouthpiece terminating said outlet section.

2. The assembly of claim 1 wherein said inlet section has a more confined airflow path at an inlet to said heater chamber than an airflow path in said heater chamber such that air flowing through said inlet section slows through said heater chamber.

3. The assembly of claim 2 wherein said plurality of elements comprises a first grinding element partially defining said grinding chamber and a second grinding element, opposed to said first grinding element, further defining said grinding chamber and rotatable with respect to said first grinding element such that contents of said grinding chamber are agitated by rotation of said second grinding element.

4. The assembly of claim 3 wherein a floor of the first grinding element is a ceiling of said heater chamber.

5. The assembly of claim 3 wherein said first grinding element is conductively coupled to said heater chamber.

6. The assembly of claim 3 wherein a floor of the first grinding element is a ceiling of said heater chamber and the second grinding element along with the first grinding element partially defines the heater chamber.

7. The assembly of claim 3 wherein the second grinding element along with the first grinding element partially defines a door of the heater chamber.

8. The assembly of claim 3 wherein said heater is mounted so as to extend transversely of said airflow path.

9. The assembly of claim 4 wherein said heater is porous.

10. The assembly of claim 1 wherein said airflow path has a smaller cross-sectional flow area just before passing through the heater than an cross-sectional flow area exiting the heater.

11. The assembly of claim 9 wherein said inlet section terminates in a wall abutting said heater, said wall having a plurality of openings whereby air flows from said plurality of openings through portions of said heater so that air at a side of said heater opposite said wall in line with said plurality of openings is cooler than air at said side which is not in line with said plurality of openings.

12. The assembly of claim 1, further comprising a casing surrounding said plurality of elements, so as to provide an insulation gap between an internal surface of said casing and said plurality of elements.

13. The assembly of claim 12 wherein the insulation gap is an air insulation gap.

14. The assembly of claim 13, wherein the plurality of elements comprises an insulator, said insulator defining a bottom of said heater chamber, said insulator having openings receiving terminals of the heater.

15. The assembly of claim 14, wherein said plurality of elements further comprises a terminal assembly disposed below said insulator and electrically connected to said terminals of said heater.

16. The assembly of claim 15, wherein at least one of said terminal assembly and said insulator has at least one opening to provide fluid communication between external air and the inlet of the heater chamber.

17. The assembly of claim 16 wherein said at least one opening provides fluid communication between the air insulation gap and the inlet of the heater chamber, whereby air drawn through said air insulation gap and into said heater chamber cools said casing.

18. The assembly of claim 3, wherein said first grinding element has a perforated floor and said second grinding element has a perforated ceiling to thereby provide axial airflow from said heater chamber through said grinding chamber.

19. The assembly of claim 16, wherein said terminal assembly comprises a connector electrically connected to said terminals of said heater, said connector for mating with an external control unit and battery for powering said heater.

20. The assembly of claim 19, wherein the connector comprises an electrically conductive pin with an axial through bore in fluid communication with said heater chamber, whereby a liquid may be injected through said through bore to said heater chamber.

21. The assembly of claim 20, wherein, the electrically conductive pin extends into an opening in a floor of the insulator.

22. The assembly of claim 1, wherein said outlet section comprises a cooling chamber, said cooling chamber having at least one hole in fluid communication with external air outside the grinding chamber, whereby a negative pressure applied at said mouthpiece results in vapours being drawn from said grinding chamber into said cooling chamber and mixed with external air drawn through said at least one hole before exiting said mouthpiece.

23. The assembly of claim 3, further comprising at least one first grinding element magnet associated with said first grinding element and at least one second grinding element magnet associated with said second grinding element for coupling with said at least one first grinding element magnet to releasably secure said second grinding element to said first grinding element.

24. The assembly of claim 23 wherein said at least one first grinding element magnet comprises a plurality of first arcuate magnets each having a first radius of curvature, said first arcuate magnets aligned end-to-end concentrically and wherein said at least one second grinding element magnet comprises a plurality of second arcuate magnets each having said first radius of curvature, said second arcuate magnets aligned end-to-end concentrically.

25. An assembly for use in an herb vaporizer, comprising:
a plurality of elements defining an air passageway with an upstream section, an expansion chamber, a grinding chamber, and a downstream section;
said upstream section terminating in said expansion chamber, said upstream section having a first cross-sectional area transverse to airflow direction at an inlet to said expansion chamber;
said expansion chamber having a second cross-sectional area transverse to said airflow direction, said second cross-sectional area being greater than said first cross-sectional area such that air flowing through said upstream section slows through said expansion chamber;
an outlet of said expansion chamber communicating to said grinding chamber;
an outlet of said grinding chamber communicating to said downstream section;
a heater disposed within said expansion chamber, said heater configured to allow passage of air therethrough; and
a mouthpiece terminating said downstream section.
